(19) United States
(12) Patent Application Publication
(10) Pub. No.: US 2025/0356965 A1
(43) Pub. Date: Nov. 20, 2025

(54) SYSTEMS AND METHODS FOR INDUCING PERSONAL REFLECTION USING A DIGITAL COMMUNICATIONS NETWORK

(71) Applicant: Sequelae, Inc., Minneapolis, MN (US)

(72) Inventors: Natalie Sheils, St. Paul, MN (US); Danielle Marria Martin, Minneapolis, MN (US); Rathish Mohan, Bothell, WA (US); Sara Taylor, Edina, MN (US); Shane Hoversten, Brooklyn Park, MN (US); Benjamin C. Wiegand, Yardley, PA (US); Steven J. Catani, Athens, GA (US)

(73) Assignee: Sequelae, Inc., Minneapolis, MN (US)

(21) Appl. No.: 19/208,057

(22) Filed: May 14, 2025

Related U.S. Application Data
(60) Provisional application No. 63/647,131, filed on May 14, 2024.

(51) Int. Cl. G16H 10/20; A61B 5/0205; A61B 5/16; G06F 16/3329

(52) U.S. Cl. CPC G16H 10/20; A61B 5/02055; A61B 5/16; G06F 16/33295

(57) ABSTRACT

Systems and methods for inducing personal reflection in an individual member using a digital communication network (DCN) are provided. The DCN is provided to a plurality of members of a population to communicate with each other. The individual member is identified based on at least one communication in the DCN from the individual member that includes at least one observation. The communication is analyzed and at least one question is generated related to the observation. The at least one question is transmitted to the individual member.

### CROSS REFERENCE TO RELATED APPLICATIONS

This application claims the benefit of U.S. Provisional Application No. 63/647,131, filed on May 14, 2024, which application is incorporated herein by reference in its entirety.

### BACKGROUND

Various embodiments relate generally to health care systems, methods, devices and computer programs and, more specifically, relate to inducing personal reflection using a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital communication networks, which can include social media where individuals can interact with others online, connect many with a community. The online community can be utilized to help build positive behaviors and encourage people to make improvements in their lives. Additionally, computers have enabled new ways to get people to view their situations.

What is needed is a way to induce user reflection through a digital communication network.

### SUMMARY

Example aspects of the present disclosure include:

A method for inducing reflection in an individual member according to at least one embodiment of the present disclosure comprises providing a digital communication network (DCN) for a plurality of members of a population to communicate with each other; identifying the individual member based on at least one communication in the DCN from the individual member that includes at least one observation related to at least one of the individual member's life, lifestyle, and health; analyzing the at least one communication in the DCN; generating, automatically, at least one question related to the at least one observation; and transmitting the at least one question to the individual member.

Any of the aspects herein, wherein transmitting the at least one question includes transmitting a request for the individual member to answer the at least one question, and wherein the method further comprises: receiving at least one answer to the at least one question from the individual member; analyzing the at least one answer; generating at least one additional question based on the at least one answer; and transmitting the at least one additional question to the individual member.

Any of the aspects herein, wherein transmitting the at least one question includes providing an incentive for the individual member to the response to the at least one question.

Any of the aspects herein, wherein transmitting the at least one question to the individual member comprises using temporal and situational cues to formulate a communication containing the at least one question.

Any of the aspects herein, wherein transmitting the at least one question to the individual member comprises asking the individual member to measure a biometric sample.

Any of the aspects herein, wherein the at least one question solicits an observation.

Any of the aspects herein, further comprising: receiving at least one of biometric data or psychometric data from the individual member through the DCN; and determining the observation based in part on at least one of the biometric data or the psychometric data.

Any of the aspects herein, wherein at least one of the biometric data and the psychometric data is derived from at least one of: a wearable device, a biometric sample, answers to a survey, and psychometric instrument.

Any of the aspects herein, wherein the biometric sample is at least one of: a saliva sample, a blood sample, a breath sample and a stool sample.

Any of the aspects herein, wherein the wearable device is an accelerometer, a HR monitor, a HRV monitor, a continuous glucose monitor, a continuous ketone monitor, and a skin temperature monitor.

Any of the aspects herein, wherein measurement of the psychometric data is made using at least one of a psychometric, psychosocial and psychological instrument.

Any of the aspects herein, further comprising sending a biometric sample kit to the individual member through the DCN to obtain the biometric sample.

Any of the aspects herein, further comprising: analyzing at least one of the biometric data or the psychometric data; and determining an efficacy of at least one of an action or an intervention based on the analysis.

Any of the aspects herein, wherein the at least one communication comprises a post by the individual member in the DCN and the post includes the at least one observation.

Any of the aspects herein, wherein the method is used in part, to facilitate or accomplish at least one of: manage the health care cost of the population; reduce the health care risk in the population; and slow the progression of an adverse health condition.

A method for inducing reflection in an individual member according to at least one embodiment of the present disclosure comprises providing a digital communication network (DCN) for a plurality of members of a population to communicate with each other;

identifying the individual member based on at least one communication in the DCN from the individual member that includes at least one observation related to at least one of the individual member's life, lifestyle, and health; analyzing the at least one communication in the DCN; generating, automatically, at least one question related to the at least one observation; transmitting the at least one question and a request for the individual member to answer the at least one question to the individual member; and receiving at least one answer to the at least one question from the individual member.

Any of the aspects herein, further comprising: analyzing the at least one answer; generating at least one additional question based on the at least one answer; and transmitting the at least one additional question to the individual member.

Any of the aspects herein, wherein transmitting the at least one question includes providing an incentive for the individual member to the response to the at least one question.

Any of the aspects herein, wherein transmitting the at least one question to the individual member comprises using temporal and situational cues to formulate a communication containing the at least one question.

Any of the aspects herein, wherein the at least one question solicits an observation.

A system to induce reflection in an individual member according to at least one embodiment of the present disclosure comprises a data repository in communication with the computer processor and storing: at least one communication having at least one observation, and at least one question, a communication analyzer which, when executed by the computer processor, analyzes the at least one communication; a digital communications network which, when executed by the computer processor, provides a network for members of a population to interact with each other; and a server controller which, when executed by the computer processor: identify an individual member based on the at least one communication in the DCN from the individual member that includes the at least one observation; analyze, using the communication analyzer, the at least one communication; generate, automatically, the at least one question related to the at least one observation; and transmit the at least one question to the individual member.

A method to improve communication, according to at least one embodiment of the present disclosure comprises providing a digital communication network (DCN) for a plurality of members of a population to communicate with each other and with one or more conversation bots; generating one or more bot communications for the one or more conversation bots to communicate with an individual member of the plurality of members; and transmitting the one or more bot communication from the one or more conversation bots to the individual member, wherein the one or more conversation bots are configured to do an associated job, and wherein the one or more bot communications are based in part on analysis of data from at least one of: an analysis of at least one communication in the DCN by the individual member, an ongoing conversation between the one or more conversation bots and the individual member, and previous conversations between the one or more conversation bots and the individual member.

Any of the aspects herein, wherein a conversation bot of the one or more conversation bots is configured to transmit at least one question designed to at least one of: induce a reflection in the individual member, understand a motivation of the individual member, collect information about the life and lifestyle of the individual member, and solicit the individual member to tell at least one of their current story and a future story.

Any of the aspects herein, wherein the reflection comprises at least one of an action, an experience, and a challenge related to the individual member.

Any of the aspects herein, wherein at least one of the action, the experience, or the challenge was at least one of actively undertaken, passively undertaken, or contemplated.

Any of the aspects herein, wherein at least one of the action, the experience, or the challenge was determined through the analysis of the at least one communication.

Any of the aspects herein, wherein each conversation bot of the one or more conversation bots has a different personality.

Any of the aspects herein, wherein the personality of the conversation bot to be used with the individual member is determined in part by the analysis of the at least one communication.

Any of the aspects herein, wherein the data was obtained from one or more communications with a subset of other members of the plurality of members.

Any of the aspects herein, wherein communication with the individual member by the one or more conversation bots is initiated based on at least one of the analysis of the at least one communication and the one or more conversation bots.

Any of the aspects herein, wherein the at least one communication references an experience that another member of the plurality of members has engaged in.

Any of the aspects herein, wherein the experience comprises at least one of: a lifestyle intervention, a dietary intervention, and a medical intervention.

Any of the aspects herein, wherein the experience is relevant to an adverse condition the individual member is experiencing.

Any of the aspects herein, wherein the communications with the conversation bot incorporate one or more excerpts of other communications on the DCN.

Any of the aspects herein, wherein the one or more excerpts comprises at least one of: post titles, post topics, quotes from the communication, and post contributors.

Any of the aspects herein, wherein the one or more conversation bots suggest at least one of a topic to the individual member to post on the DCN, at least one of an action or an experience the individual member can undertake to aid in future reflection, and a topic for the individual member to reflect on, and wherein the one or more conversation bots provides access to a tool to at least one of help the individual member create the post and to enhance a reflection about an action.

Any of the aspects herein, wherein the tool comprises at least one of a biosample, a wearable device, a psychometric instrument, and a structured activity.

Any of the aspects herein, wherein at least one of the topic, the action, and the experience is determined by an analysis of one or more communications by other members of the plurality of members.

Any of the aspects herein, wherein the bot communication between the one or more conversation bots and the individual member is via at least one of text messages, voice conversation, and in virtual reality.

Any of the aspects herein, wherein the method is performed to, at least one of: lower a health risk of the population, and manage a disease.

A system to improve communication according to at least one embodiment of the present disclosure comprises a computer processor; a data repository in communication with the computer processor and storing: one or more communications, one or more bot communications; one or more conversation bots; a digital communications network which, when executed by the computer processor, provides a network for members of a population to interact with each other; and a server controller which, when executed by the computer processor: generate the one or more bot communications; transmit the one or more bot communications from the one or more conversation bots to an individual member, wherein the bot communication is based in part on analysis of data from at least one of: an analysis of at least one communication in the DCN by the individual member, an ongoing conversation between the one or more conversation bots and the individual member, and previous conversations between the one or more conversation bots and the individual member.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Z₀, the phrase is intended to refer to a single element selected from X, Y, and Z, a combination of elements selected from the same class (e.g., X₁ and X₂) as well as a combination of elements selected from two or more classes (e.g., Y₁ and Z₀).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4A shows an example of a computing system, in accordance with one or more embodiments.

FIG. 4B shows an example of a network, in accordance with one or more embodiments.

### DETAILED DESCRIPTION

Various embodiments are directed to inducing reflection in an individual member of a population within a digital communication network (DCN). Reflections are an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

Using a conversation bot, the DCN is able to help patients with seeking help for medical conditions. The conversation bot, using artificial intelligence (AI), can review community discussions on the DCN to identify key issues that may be a concern for an individual member. The bot can ask questions of the individual member so that they can reflect on their situation and/or better appreciate the consequences of any actions. This can help identify conditions, reactions, etc. The conversation bot can also review community discussions to identify possible questions that can, for example, be asked of a doctor to help a patient (e.g., the individual member) with the preliminary work and setting them up to get the key information for proper medical intervention and treatment.

Various embodiments use AI-driven bots to induce more user reflections. In traditional systems, conversation bots respond to questions with community or expert-sourced answers whereas in embodiments of the present disclosure the conversation bots respond to observations (which may be provided directly or indirectly by the user) with questions designed to cause user reflection on those observations.

User reflections may be used by the conversation bot to pinpoint better information for the user, for example, by providing more applicable community or expert-sourced answers. In other embodiments, the individual member reflections may be provided to care givers and/or physicians in order to better evaluate the individual member.

Feedback, interventions, or suggestions to the individual member can be generated and provided, and such feedback, interventions, or suggestions can be based in part on creating greater awareness of lifestyle changes and medial options. A lifestyle (or behavior) change can be evaluated based on the individual member's reflections. Such induced reflections may be used to help identify the individual member's condition, for example, to evaluate whether a lifestyle change is working.

Additionally, the individual member's reflections can be used to provide the individual member's experience as a social aspect. As a social process, value can be placed on those members of a community that have relevant experience. The reflections from the individual members can be induced in order to help elevate the aspects of the experience that may be more instructional to the community as a whole. This can help increase the number of members of the community thinking about an action, for example, creating an atmosphere where people can learn about others in who have undergone a change they are considering.

The system can also help an individual member in creating posts about their experiences. The AI-driven conversation bots can engage the individual member and propose content for the post based on the individual member's response. The engagement may include inducing reflections and/or analysis of the individual member's health situation. The posts may be for one-to-one communications, such as facilitating the conversation with the conversation bot, or one-to-many communications, such as general posts for the population.

In some embodiments, the conversation bot may also propose content for the post based on other communications in the DCN. By proposing content, the bots can reduce friction for the individual member in creating communications for the DCN. By facilitating such posts, the DCN can increase general awareness in population of health conditions (or risks).

Additionally, the community can be used to help support the individual member in other ways. Lifestyle interventions are inherently safe. They use the philosophy that any action now is preferred to a “better” action later. They also support the concept that ideas and communication are healthcare.

The community can also be used to encourage lifestyle interventions, which are inherently safe. Such community may use the philosophy that any action now is preferred to a “better” action later and also support the concept that ideas and communication are healthcare. Conventionally, lifestyle change has been looked at as in individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual member or members of a population. For example, in a community where a person is a peer, the actions they take and learn from are that their volition and may result in increased agency (or autonomy) or self-efficacy. This not only increase the chances of continuous lifestyle improvement, but improved outcomes throughout the heath system.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Thus, it is desirable to provide systems and methods that induce reflection in an individual member whether through the DCN directly or via one or more conversation bots. Such reflections can beneficially provide insight into the individual member's life and lifestyle and whether certain actions or interventions are beneficial to the individual member.

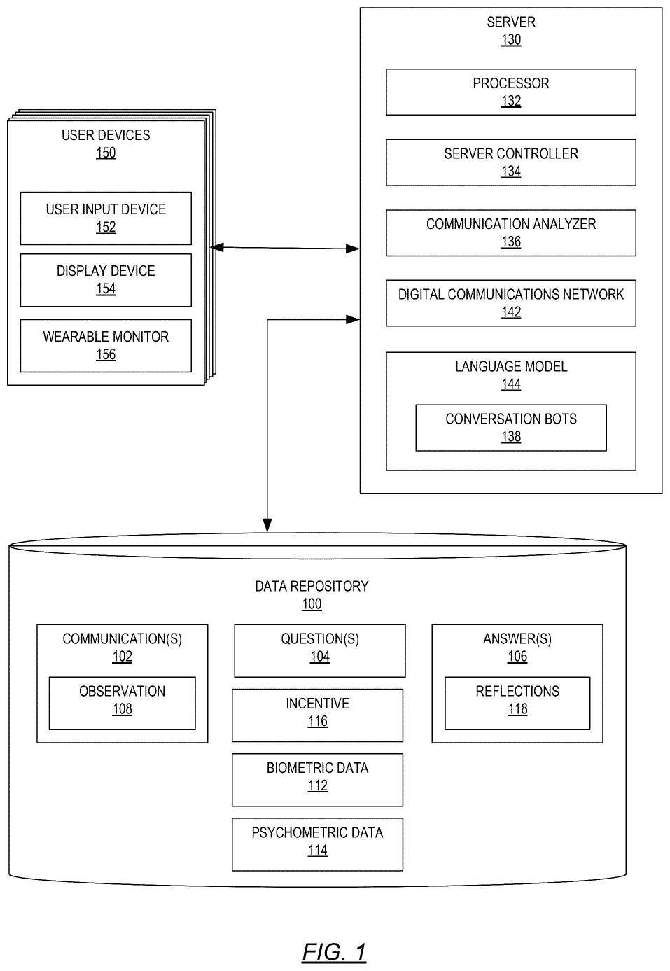

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (102). The communications (102) are communications from members in a digital communication network (DCN) (142) (described in more detail below). The communications (102) may be, for example, text-based, image-based, multimedia communications, audio communications, or any combinations thereof. The communications may be between members, communications in a forum, communications with the DCN (142), or communications generated by the DCN (142) (e.g., communications from a conversation bot, also referred to as a chat bot).

The communications (102) in the DCN (142) by an individual member may include one or more observations (108). The one or more observations (108) may relate to at least one of the individual member's life, lifestyle or health. The observations (108) may be based in part on biometric data and/or psychometric data, which will be discussed in more detail below. The communications (102) may include, for example, a post and/or a post title that the individual member created in the DCN (142).

The communications (102) may also be referred to as bot communications when the communications are generated and received from one or more conversation bots (138).

The data repository (100) also stores question(s) (104). The questions (104) are questions directed to the individual member and may include yes/no questions or open-ended questions. The questions (104) may be designed to induce a reflection in the individual member, understand a motivation of the individual member, collect information about the life and lifestyle of the individual member, and solicit the individual member to tell at least one of their current story and a future story. The questions (104) can include one question, two questions, or more than two questions. The questions (104) may also include a request for the individual member to answer the question (104). The request may include buttons (e.g., yes/no buttons), a text and/or media box for the individual member to type in or attach media.

The data repository (100) also stores incentives (116). The incentives (116) are discounts, coupons, money, or other rewards to incentivize the individual member to answer the questions (104). The incentives (116) may be, for example, a discount or may cover the cost for an activity or an item. The incentive (116) may include one or more incentives (116).

The data repository (100) also stores answer(s) (106). The answers (106) are answers (106) to the questions (104) received from the individual member. The answers (106) may include, for example, a yes/no answer, text, and/or media (e.g., audio, visual, images, videos, etc.). The answers (106) can be used to generate additional or follow up questions (104). The additional or follow up questions can be used to, for example, solicit additional observations (108).

The answers (106) may also include one or more reflections (118). The reflections (118) may be a reflection of an action, an experience, and/or a challenge related to the individual member. The action, the experience, and/or the challenge may have been actively undertaken, passively undertaken, or contemplated and can be determined from an analysis of the communications (102) and/or the answers (106).

The data repository (100) also stores biometric data (112). The biometric data (112) may be derived from, for example, a wearable device, a biometric sample, answers to a survey, and/or a psychometric instrument. When the biometric data (112) is a biometric sample, the biometric sample may be, for example, a saliva sample, a blood sample, a breath sample and/or a stool sample.

Biometric data (112) can be used in many instances such as the provisioning and commissioning of healthcare. For example, biometric data (112) can be used to diagnose diabetes, prescribe hypertension medication with a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data (112)'s measurement, as well as the cost-benefit ratio of the intervention compared with other interventional options.

The data repository (100) also stores psychometric data (114). The psychometric data (114) is data derived from the individual member and can be used to forecast or predict the individual member's behavior. The psychometric data (114) may also be derived from, for example, for example, a wearable device, a biometric sample, answers to a survey, and/or a psychometric, psychosocial, and/or psychometric instrument.

The biometric data (112) and the psychometric data (114) can also be used in a “feedback” decision tree where, for example, an intervention or treatment can be prescribed or recommended to an individual member. Then the biometric data (112) and the psychometric data (114) can be obtained to study the effects or results of the intervention or treatment.

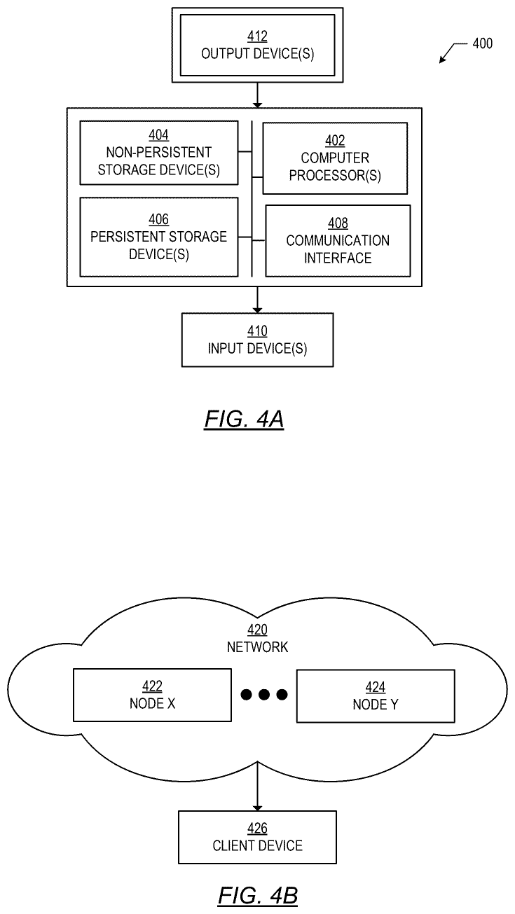

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 4A and FIG. 4B.

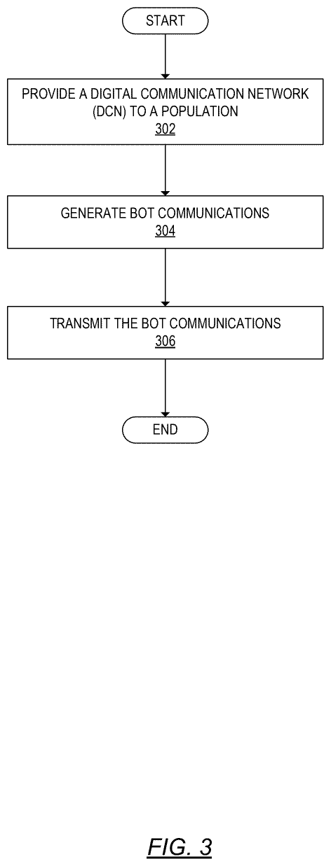

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (302) of FIG. 3A.

The server (130) also may include a server controller (134). The server controller (134) is software or application specific hardware which, when executed by the computer processor (132), controls and coordinates operation of the software or application specific hardware described herein. Thus, the sever controller (134) may control and coordinate execution of the communication analyzer (136) and the language model (144).

The server (130) also includes a communication analyzer (140). The communication analyzer (140) is software or application specific hardware which, when executed by the computer processor (132), receives the communications (102) as input and can generate the questions (104) based on the observations (108). The communication analyzer (140) can also generate communications (102) for the conversation bots (138) to use to communicate with the individual member.

The server (130) also includes a digital communications network (DCN) (142). The DCN (142) is a network through which members of a population can interact with each other, or with a system supported by the DCN. For example, an individual member can interact with the DCN (142) by receiving questions (104) and a request to answer the questions (104) and submitting their answer (106) to the questions (104) to the DCN (142). The DCN (142) can use the answer(s) (106) for a variety of purposes such as, for example, generating new or additional questions (104).

The DCN (142) can also manage an individual member's communication feed to prioritize messages which contain ideas that might be applicable to them. This can be based on various factors, such as the individual member's own biological information, the individuals they follow in the network, etc.

The DCN (142) can also provide means for members of the population to communicate with each other. For example, the individual member can share their communications (102) that may include observations (108) and/or reflections (118) or any other communication with other members in the DCN (142). Communications between the members of the population in the DCN (142) can be, for example, one to one, one to many, one to the system, the system to one, or the system to many. Further, in some embodiments, the DCN (142) may use artificial intelligence (AI) to derive communications from the DCN (142) from an analysis of communications in the DCN (142) or from biometric data (112) and/or psychometric data (114) provided to the DCN (142) from the members.

The server (130) also includes a language model (144). The language model (144) is a natural language processing machine learning model. An example of the language model (144) may be a large language model, such as CHATGPT® or LLAMA®. However, many different language models may be used. In some embodiments, the language model (144) can power the one or more conversation bots (138) to communication with the members of the population.

The conversation bots (138) can communicate with the individual member in discrete instances or continuously. The conversation bot (138) may also focus it interactions with an individual member on a topic for a period of time. The period of time could be a day, a week, a month, etc. The period of time may instead be a number of conversations with the member. The conversation bot (138) can also generate communications for the individual member to, for example, post in the DCN (142). In such embodiments, the communications created for the individual member by the conversation bot (138) may be designed to mimic the individual member's style. The communications crated by for the individual member by the conversation bot (138) may alternatively be formulated in a style designed to provide a positive reaction in community members near the individual member in a social graph of the DCN (142). The communications crated by for the individual member by the conversation bot (138) may be formulated in a style designed to provide a positive reaction in community members who have an adverse health condition associated with the communication.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send stress indicator data (110) to the server (130). In an alternative embodiment, a separate wearable device may be in communication with the user device (150), such as a smart watch, a HR monitor, a HRV monitor, a continuous glucose monitor, a continuous ketone monitor, a skin temperature monitor, and/or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

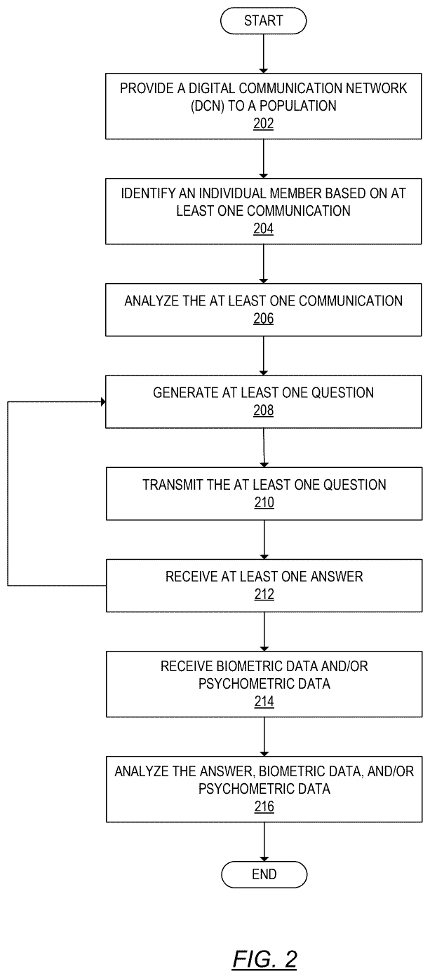

FIG. 2 is a logic flow diagram that illustrates a method, and a result of execution of computer program instructions, in accordance with various embodiments. The method can be used to induce or encourage reflection in an individual member. The method can also, in other instances, be used in part to facilitate or accomplish at least one of: manage the health care cost of the population; reduce the health care risk in the population; and/or slow the progression of an adverse health condition.

At Block 202, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots such as the one or more conversation bots (138).

At Block 204, a step of identifying an individual member based on at least one communication is provided. The communication may be the same as or similar to the communication (102) and can include an observation such as the observation (108). The observation may be related to at least one of the individual member's life, lifestyle, and health. In some embodiments, the communications may be a post by the individual member in the DCN and the post includes the at least one observation.

At Block 206, a step of analyzing the at least one communication is provided. The communication may be analyzed by, for example, a communication analyzer such as the communication analyzer (136). The communication analyzer may identify the observation within the communication. In embodiments where the communication is an answer such as the answer (106), the communication analyzer may identify one or more reflections such as the reflections (118) in the answer.

At Block 208, a step of generating at least one question is provided. The at least one question may be the same as or similar to the question (104). The at least one question may be generated automatically and may be based on the observations identified in the individual member's communications.

At Block 210, a step of transmitting the at least one question to an individual member of the population is provided. The question may be transmitted to, for example, a user device such as the user device (150) by the DCN. Transmitting the question may also include providing an incentive such as the incentive (116) for the individual member to the response to the at least one question. Transmitting the question may also include transmitting the at using temporal and situational cues to formulate a communication containing the at least one question.

In some embodiments, transmitting the question may include asking the individual member to measure or provide biometric data and/or psychometric data. As previously described, the biometric data and/or the psychometric data is derived from at least one of: a wearable device, a biometric sample, answers to a survey, and psychometric instrument. In such embodiments, a biometric sample kit may be provided or sent to the individual member via the DCN to obtain the biometric sample.

At Block 212, a step of receiving at least one answer to the at least one question is provided. The at least one answer may be the same as or similar to the answer (106). The question may be transmitted by, for example, the user device to the DCN. The answer may be answered using asynchronous communication or in a synchronous communication with a conversation bot.

The question and the answer may be asked and answered, respectively, over a defined time-period. In some embodiments, the time-period is an hour, a day, a week, a month, several months, or any other increment of time. The question and/or the answer may also be provided verbally and any verbal question or answer may be transcribed automatically to text.

It will be appreciated that the Block 212 can result in repeating at least the Blocks 208 and 210. In other words, the answer may be used to generate an additional question or a follow up question based on the answer. The additional question or follow up question may be designed or used to solicit more information about the observation or may be used to induce or encourage the observation.

At Block 214, a step of receiving biometric data and/or psychometric data is provided. The biometric data may be the same as or similar to the biometric data (112) and the psychometric data may be the same as or similar to the psychometric data (114). The biometric data and/or the psychometric data may be received from the individual member through, for example, the DCN.

At Block 216, a step of analyzing the answer, the biometric data, and/or the psychometric data is provided. Such analysis can be useful to determine an efficacy of an intervention (or other activity) and to detect benefits that would otherwise be within the variance of a population-controlled sample. The analysis can also be useful in assessing the impact of lifestyle changes in the individual member. For example, the individual member may reflect on how an intervention is resulting in them feeling better or increasing their energy, which indicates that the intervention is successful.

In another example, the analysis can be used to manage a variety of conditions such as, for example, chronic system inflammation (CSI). For example, specific inflammatory markers may be of interest, e.g., in diagnosing and approving drugs to treat rheumatoid arthritis (RA). Such markers may include, for example, C-reactive protein (CRP), which can be combined with other markers. Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

By way of background, generalized CSI is not specific to any disease and, as such, does not often fit into a single category in a healthcare system. Such characterization results in CSI not being treated in a risk/benefit positive way with drugs or surgery or with any of the tools the provider and insurer community provide. Instead, it is driver and marker of disease progression in general, even if the particular disease is unknown. While the risk-benefit-ratio of drugs and surgery are not positive it can be improved (and therefore disease progression can be slowed) with lifestyle, social situation, emotional management with little or no risk.

Although CSI effects progression of a disease, progression is not typically measured in healthcare. Inflammation and its management impact disease progression and, at some point, disease state. If disease progression can be slowed, the disease state can be avoided and the population is less sick. Thus, it is beneficial to track CSI and whether an intervention has improved or not improved CSI through the individual member's communications and reflections in the DCN.

The method described in FIG. 2 can be useful in many scenarios. For example, health care in a population can be improved by providing a means for members of said population to reflect on their health through a DCN. The communications in the DCN are analyzed to determine questions for a user which are intended to help that user reflect. The DCN can then send the message (or messages) to the user. In some cases, the process can be iterative, where the user's responses to the questions may generate additional questions.

The method described in FIG. 2 can include more or less steps. One or more steps or any combination of steps may also be repeated in the method described in FIG. 2.

FIG. 3 is a logic flow diagram that illustrates a method, and a result of execution of computer program instructions, in accordance with various embodiments. The method can be used to improve communications in, for example, a DCN. In other embodiments, the method is performed to, at least one of: lower a health risk of the population, and/or manage a disease.

At Block 302, a step of providing a digital communication network (DCN) to a population is provided. The Block 302 is generally the same as or similar to the Block 202 of FIG. 2 described above.

At Block 304, a step of generating one or more bot communications is provided. The one or more bot communications may be the same as or similar to the communications (102). The bot communications may be based in part on analysis of data from an analysis of the at least one communication in the DCN, an ongoing conversation between the one or more conversation bots and an individual member, and previous conversations between the one or more conversation bots and the individual member. The data can be obtained from one or more communications with a subset of other members of the plurality of members.

In some embodiments, the at least one communication can reference an experience that another member of the plurality of members has engaged in. The experience can include, for example, a lifestyle intervention, a dietary intervention, and a medical intervention. The experience can also be relevant to an adverse condition the individual member is experiencing. The adverse condition could be any of: a chronic systemic inflammation, malaise or low energy, a disease (such as, Obesity, Diabetes, RA, Crohn's, Psoriasis, eczema, CVD, CHF, COPD, asthma, depression, anxiety, etc.), a health risk (such as, falling, becoming infected, etc.), a social dysfunction and/or a prodromal disease.

At Block 306, a step of transmitting the bot communications is provided. The bot communication may be transmitted to the individual member by a conversation bot of one or more conversation bots such as the conversation bot (138). The bot communication can include, for example, text messages, voice conversations, virtual reality, or a combination thereof.

In some embodiments, the bot communication includes a question such as the question (104). In such embodiments, a conversation bot of the one or more conversation bots is configured to transmit the question and the question is designed to at least one of: induce a reflection such as the reflection (118) in the individual member, understand a motivation of the individual member, collect information about the life and lifestyle of the individual member, and/or solicit the individual member to tell at least one of their current story and a future story. In some embodiments, the reflection includes an action, an experience, and/or a challenge related to the individual member. The action, the experience, and/or the challenge may be actively undertaken, passively undertaken, or contemplated by the individual member. The action, the experience, and/or the challenge may have also been determined through the analysis of the communication from the individual member.

The one or more conversation bots may include one conversation bot, two conversation bots, or more than two conversation bots. Each conversation bot is also configured to do an associated job, and each conversation bot may also have a different personality. The associated job may be, for example, facilitate reflections, find useful communications, say good morning and inspire a positive start to a day, handle routine network tasks, provide custom communication summaries. The personality of the conversation bot to be used with the individual member may be determined in part by the analysis of the communication from the individual member. Such analysis may be useful in determining a personality of the individual member so that a matching or complimentary personality of the conversation bot can be selected. The personality of the conversation bot can also change as the conversation changes. For example, when attempting to induce a reflection, the conversation bot may use a first personality, then switch to a second personality when helping draft a post regarding such reflection.

In some embodiments, communication with the individual member by the one or more conversation bots is initiated based on the analysis of the at least one communication from the individual member and can be initiated by the one or more conversation bots. The communications with the conversation bot by the individual member can incorporate one or more excerpts of other communications in the DCN such as communications from other members in the DCN. The excerpts can include, for example, post titles, post topics, quotes from the communication, and post contributors. The excerpts can also include summaries of communications on the DCN. These summaries may be created automatically by the DCN and/or are based on a topic, a member's communications, or a time period.

The one or more conversation bots may also suggest at least one of a topic to the individual member to post on the DCN, at least one of an action or an experience the individual member can undertake to aid in future reflection, and/or a topic for the individual member to reflect on. In such instances, the one or more conversation bots may provide access to a tool to help the individual member to create the post and/or to enhance a reflection about an action. The tool can include, for example, a biosample, a wearable device, a psychometric instruction, and/or a structured activity. In such instances, the topic, the action, and the experience can be determined by an analysis of one or more communications by other members of the plurality of members. In such embodiments, the topic may be relevant to one or more adverse condition the member is experiencing. The conversation bot may also create, in part, a communication for the member to post on the DCN.

In some embodiments, the bot communication may be transmitted via virtual reality (VR). In such embodiments, the use of VR can facilitate reflection, and a conversation bot may be used in VR to facilitate reflection on the interventions in the VR environment. As discussed above, the conversation bot may be given a specific personality or role. In VR, the bot may also be provided with an appearance based on the personality or role. Additionally, data from the VR intervention can be used to inform the reflection dialog.

In at least one example of the method described above in FIG. 3, the method beneficially provide means for an individual member to communicate their health journey with the rest of the healthcare system, particularly their primary care physician. These communications can be automatically generated from the member's interactions with the DCN, including posts, experiences, biometric results, discussions and the like. Bot communications (or any communication) can be triggered for production when the individual member indicates (directly or indirectly) that they have an upcoming interaction with the healthcare system. This can include providing questions for the individual member to ask their physician. In this manner, the DCN, through a bot, causes the user to reflect and then assists the individual member during a meeting with the physician by providing the potential questions to ask.

In another example, during communication between the individual member and the conversation bot, the DCN can analyze the individual member's responses. Based on the input from the individual member, the DCN may identify an adverse health condition for the individual member. The conversation bot may then gather more information regarding the adverse health condition, for example, to develop more details to confirm the adverse health condition. The communication may be based on related experiences from another member of the population. The conversation bot may also quote content from a post by that other member. The experience may be a lifestyle intervention, a dietary intervention, a medical intervention (even one that was unplanned), etc. The experience may also be relevant to one or more adverse conditions that the member is experiencing.

Using analysis of the individual member's communications in the DCN, the conversation bot can suggest a reflection on a topic in a one-top-one communication. The suggestion may be made as part of a question to the individual member. The conversation bot may then focus its conversations with that individual member on that topic for a period of time (e.g., a number of exchanges, a few minutes, a few days, etc.). The conversation bot may prompt the user to reflect on various classes of action. The action may be active or passive. The action may also be one that was contemplated even if not undertaken.

The method described in FIG. 3 can include more or less steps. One or more steps or any combination of steps may also be repeated in the method described in FIG. 3.

The methods described above in FIGS. 2 and 3 in which reflection is induced in an individual member (whether through the DCN or a conversation bot) beneficially allow evaluation of various activities without increasing healthcare system cost. This can also be used to improve community awareness of interventions. In some situations, biometric tools can also be provided directly to the users to help assess if their actions are working.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method for inducing reflection in an individual member, the method comprising:
providing a digital communication network (DCN) for a plurality of members of a population to communicate with each other;
identifying the individual member based on at least one communication in the DCN from the individual member that includes at least one observation related to at least one of the individual member's life, lifestyle, and health;
analyzing the at least one communication in the DCN;
generating, automatically, at least one question related to the at least one observation; and
transmitting the at least one question to the individual member.

2. The method of claim 1, wherein transmitting the at least one question includes transmitting a request for the individual member to answer the at least one question, and wherein the method further comprises:
receiving at least one answer to the at least one question from the individual member;
analyzing the at least one answer;
generating at least one additional question based on the at least one answer; and
transmitting the at least one additional question to the individual member.

3. The method of claim 1, wherein transmitting the at least one question includes providing an incentive for the individual member to the response to the at least one question.

4. The method of claim 1, wherein transmitting the at least one question to the individual member comprises using temporal and situational cues to formulate a communication containing the at least one question.

5. The method of claim 1, wherein transmitting the at least one question to the individual member comprises asking the individual member to measure a biometric sample.

6. The method of claim 1, wherein the at least one question solicits an observation.

7. The method of claim 1, further comprising:
receiving at least one of biometric data or psychometric data from the individual member through the DCN; and
determining the observation based in part on at least one of the biometric data or the psychometric data.

8. The method of claim 7, wherein at least one of the biometric data and the psychometric data is derived from at least one of: a wearable device, a biometric sample, answers to a survey, and psychometric instrument.

9. The method of claim 8, wherein the biometric sample is at least one of: a saliva sample, a blood sample, a breath sample and a stool sample.

10. The method of claim 8, wherein the wearable device is an accelerometer, a HR monitor, a HRV monitor, a continuous glucose monitor, a continuous ketone monitor, and a skin temperature monitor.

11. The method of claim 8, wherein measurement of the psychometric data is made using at least one of a psychometric, psychosocial and psychological instrument.

12. The method of claim 8, further comprising sending a biometric sample kit to the individual member through the DCN to obtain the biometric sample.

13. The method of claim 7, further comprising:
analyzing at least one of the biometric data or the psychometric data; and
determining an efficacy of at least one of an action or an intervention based on the analysis.

14. The method of claim 1, wherein the at least one communication comprises a post by the individual member in the DCN and the post includes the at least one observation.

15. The method of claim 1, wherein the method is used in part, to facilitate or accomplish at least one of: manage the health care cost of the population; reduce the health care risk in the population; and slow the progression of an adverse health condition.

16. A method for inducing reflection in an individual member, the method comprising:
providing a digital communication network (DCN) for a plurality of members of a population to communicate with each other;
identifying the individual member based on at least one communication in the DCN from the individual member that includes at least one observation related to at least one of the individual member's life, lifestyle, and health;
analyzing the at least one communication in the DCN;
generating, automatically, at least one question related to the at least one observation;
transmitting the at least one question and a request for the individual member to answer the at least one question to the individual member; and
receiving at least one answer to the at least one question from the individual member.

17. The method of claim 16, further comprising:
analyzing the at least one answer;
generating at least one additional question based on the at least one answer; and
transmitting the at least one additional question to the individual member.

18. The method of claim 16, wherein transmitting the at least one question includes providing an incentive for the individual member to the response to the at least one question.

19. The method of claim 16, wherein transmitting the at least one question to the individual member comprises using temporal and situational cues to formulate a communication containing the at least one question.

20. A system to induce reflection in an individual member, the system comprising:
a computer processor;
a data repository in communication with the computer processor and storing:
at least one communication having at least one observation, and
at least one question,
a communication analyzer which, when executed by the computer processor, analyzes the at least one communication;
a digital communications network which, when executed by the computer processor, provides a network for members of a population to interact with each other; and
a server controller which, when executed by the computer processor:
identify an individual member based on the at least one communication in the DCN from the individual member that includes the at least one observation;
analyze, using the communication analyzer, the at least one communication;
generate, automatically, the at least one question related to the at least one observation; and
transmit the at least one question to the individual member.
